(19) United States
(12) Patent Application Publication
(10) Pub. No.: US 2019/0254325 A1
(43) Pub. Date: Aug. 22, 2019

(54) ENHANCED KAVA EXTRACT, METHOD OF MAKING THE SAME, AND COMPOSITIONS THEREOF

(71) Applicant: Monsoon Beverages LLC, Billings, MT (US)

(72) Inventor: Michael Munsell, Billings, MT (US)

(21) Appl. No.: 15/899,385

(22) Filed: Feb. 20, 2018

(51) Int. Cl. A23L 33/105; A23L 5/20; A23L 2/52; A23G 3/48; A61K 36/67

(52) U.S. Cl. CPC A23L 33/105; A23L 5/23; A23L 2/52; A23G 3/48; A23G 2200/14; A23V 2002/00; A23V 2300/14; A23V 2300/44; A23V 2250/2128; A61K 36/67

(57) ABSTRACT

Embodiments herein relate to a human edible or drinkable product comprising an enhanced kava extract, wherein the enhanced kava extract comprises a kava product extracted using a material comprising CO2 and/or ethanol extraction method from a selected part of a kava plant and containing 50% or more of a kavalactone as measured by HPLC, wherein the kava plant is not a wild type kava such that the kava product is safe for human consumption, wherein the composition further comprises an additive comprising a tetrahydrocannabinol extract, a cannabidiol extract, a hemp extract or combinations thereof. In one embodiment, the enhanced kava extract comprises a kava product extracted using a non-organic solvent.

### BACKGROUND

U.S. Pat. No. 5,770,207A reveals a dietary supplement of the invention comprises Kava root extract and at least one additional relaxing herb selected from the group consisting of Passion Flower, Chamomile Flowers, Hops, and Schizandra Fruit.

U.S. Pat. No. 5,976,550A reveals a composition of matter to achieve a fat reducing effect comprising: a sugar based confectionary to be eaten before a meal to minimize the appetite, a therapeutic amount of chitosan mixed in said confectionary together with a therapeutic amount of kava whereby said chitosan functions to attract fat to form a non-digestible amalgam of chitosan and fat that passes out of the body and whereby said kava functions to reduce a desire to eat by mildly anesthetizing the mouth.

U.S. Pat. No. 9,636,373B1 reveals to a kava-based beverage composition. In one embodiment, a beverage composition includes water; kava extracts, milk thistle extract and yerba mate extract.

U.S. Pat. No. 8,383,169 B1 reveals a composition comprises kava root extract and at least one additional component of lemon balm or chamomile extract to serves as a relaxant and mind energizer that affords the benefit of reduction in the incidence or severity of stomach upset and/or hangover.

### SUMMARY OF THE INVENTION

The present invention discloses a composition for beverages, aerated beverages, candies, hard and chewable gummy candies, confectionaries, and effervescent powder or tablet and dietary supplement comprising enhanced kava extract and at least one additional component of cannabis and/or hemp extract.

The present invention also discloses the process to prepare the composition is also disclosed according to present invention.

The disclosed composition of the present invention comprises an enhanced kava extract which is derived from kava plants and the enhanced kava extract produces a state of calmness, relaxation and well-being without diminishing cognitive performance or negative side effects.

Owing to their ability to provide relief to an array of symptoms including pain, nausea, seizures, spasms and inflammation, the Cannabis containing compositions of the embodiments herein are used for both medical use and recreational use. Cannabis containing compositions are used to treat chronic pain and muscle spasm for medical use but have common side effects include but not limited to dizziness, feeling tired, vomiting, anxiety, agitated restlessness and hallucinations.

In one embodiment, the disclosed composition according of the present invention comprises an enhanced kava extract that diminishes, suppresses or relieves the common negative side effects cannabis or hemp extract or cannabis containing compositions by serving as a relaxant, providing relief from anxiety, providing calming benefits, producing a state of calmness, relaxation and well-being without diminishing cognitive performance.

In one embodiment, kavalactones in the enhanced kava extract are main source or the principal ingredients for serving as a relaxant, providing relief from anxiety, providing calming benefits, relief from nausea producing a state of calmness, relaxation and well-being without diminishing cognitive performance. There are different types of kavalactones present in the kava plant and six of these kavalactones make up greater than 95% of the total kavalactones pharmacological activity.

In one embodiment, the quality of the enhanced kava extract is related to the higher amounts of kavalactone content, higher concentration of kavalactone content, favorable compositional mix of kavalactone. In another embodiment, the quality of the enhanced kava extract is related to the higher amounts of six major kavalactone content, higher concentration of six major kavalactone content, and favorable compositional mix of six major kavalactones.

An embodiment relates to a composition comprising an enhanced kava extract, wherein the enhanced kava extract comprises a kava product extracted using a material comprising CO2 and/or ethanol extraction method from a selected part of a kava plant and containing 50% or more of a kavalactone as measured by HPLC, wherein the kava plant is not a wild type kava such that the kava product is safe for human consumption.

Preferably, the selected part of the kava plant comprises a lower part of the basal stem, rhizomes and/or roots.

Preferably, the material comprises supercritical CO2.

Preferably, the material comprises supercritical CO2 and ethanol.

Preferably, the enhanced kava extract contains 60% or more of the kavalactone as measured by HPLC.

Preferably, the composition further comprises an additive comprising a tetrahydrocannabinol extract, a cannabidiol extract, a hemp extract or combinations thereof. These extracts could be made by a variety of methods, including using extraction using ethanol, CO2, H2O, BHO, RSO, and rosin as a solvent.

BHO is a waxy concentrated cannabis extract made by pushing liquid butane (which liquefies easily) through a tube packed with frosty buds. The resulting solution is a mix of concrete oils, waxes, cannabinoids, terpenoids, and sometimes chlorophyll. Butane is a nonpolar chemical solvent made from petroleum and natural gas.

RSO is Rick Simpson Oil; it is a full extract cannabis oil meant to be taken orally or applied topically. RSO is a marijuana extract made utilizing a solvent to extract cannabinoids. The most common solvent used to produce RSO is grain alcohol, but some other solvents like ethanol or butane are sometimes used. Flower (bud) material is placed in a large container and alcohol is added. The entire mixture is stirred and crushed into the alcohol. After a time, the alcohol is drained from the remaining plant material. That mixture is then heated in a container, such as a rice cooker, so that the residual alcohol evaporates. The end product is a high potency oil often dark in color with a thick consistency. RSO can be siphoned into a syringe style applicator for dosing which offers the advantage of a long shelf life as oxidation does not easily occur.

Preferably, the additive consists essentially of the tetrahydrocannabinol extract.

Preferably, the additive consists essentially of the cannabidiol extract.

Preferably, the additive consists essentially of the hemp extract.

Preferably, the composition provides a benefit of reduction in an incidence or severity of anxious feelings or panic sometimes associated with the tetrahydrocannabinol.

Preferably, the composition is selected from the group consisting of a hard candy, a chewable candy, a beverage, a dietary supplement, and combinations thereof.

Preferably, hard candy, the chewable candy, the beverage or the dietary supplement comprises about 5 mg-950 mg of the enhanced kava extract in the total candy and about 1 mg-900 mg of a tetrahydrocannabinol extract in the total candy.

Preferably, hard candy, the chewable candy, the beverage or the dietary supplement comprises about 5 mg-950 mg of the enhanced kava extract in the total candy and about 1 mg-900 mg of a cannabidiol extract in the total candy.

Preferably, the hard candy, the chewable candy, the beverage or the dietary supplement comprises about 5 mg-950 mg of the enhanced kava extract in the total candy and about 1 mg-900 mg of a hemp extract in the total candy.

An embodiment relates to a composition comprising an enhanced kava extract, wherein the enhanced kava extract comprises a kava product extracted using a non-organic solvent from a selected part of a kava plant and containing 50% or more of a kavalactone as measured by HPLC, wherein the kava plant is not a wild type kava such that the kava product is safe for human consumption; wherein the non-organic solvent comprises supercritical CO2.

Another embodiment relates to a method of making a composition comprising an enhanced kava extract, wherein the enhanced kava extract comprises a kava product extracted using a material comprising CO2 and/or ethanol extraction method from a selected part of a kava plant and containing 50% or more of a kavalactone as measured by HPLC, wherein the kava plant is not a wild type kava such that the kava product is safe for human consumption, the method comprising exposing the selected part of the kava plant to the material and extracting the kava product.

The method could further comprise post extraction recovery and/or concentration of the kava product.

Preferably, the selected part of a kava plant comprises a lower part of the basal stem, rhizomes and/or roots.

Preferably, material consists essentially of liquid supercritical CO2.

Preferably, the material consists essentially of ethanol.

Preferably, the enhanced kava extract contains 70% or more of the kavalactone as measured by HPLC.

Preferably, the enhanced kava extract contains 80% or more of the kavalactone as measured by HPLC.

In one embodiment, the enhanced kava extract is considered to be enhanced for having higher amounts of kavalactone, higher concentration of kavalactone content, and favorable compositional mix of six major kavalactones. In another embodiment, the enhanced kava extract is considered to be enhanced for having higher amounts of six major kavalactone content, higher concentration of six major kavalactone content, and favorable compositional mix of six major kavalactones.

In another embodiment, the enhanced kava extract is considered to be enhanced for consisting of at least 50% of the six major kavalactones with higher pharmacological activity. In another embodiment, the enhanced kava extract comprises at least 50% of the six major kavalactones with higher pharmacological activity.

In one embodiment, the ability to produce a state of calmness, relaxation and well-being, the ability to serve as a relaxant. provide relief from anxiety, provide relief from nausea, provide calming benefits are dependent on the concentration of the six major kavalactones in the enhanced kava extract, the amounts of six major kavalactones in the enhanced kava extract and favorable compositional mix of the six major kavalactones in the enhanced kava extract. In another embodiment, the ability to produce a state of calmness, relaxation and well-being, the ability to serve as a relaxant. provide relief from anxiety, provide relief from nausea, provide calming benefits are dependent on the compositions of the enhanced kava extract which comprises higher amounts of kavalactone, higher concentration of kavalactone content, and favorable compositional mix of six major kavalactones.

In one embodiment, the effectiveness to produce a state of calmness, relaxation and well-being is dependent on the enhanced kava extract comprising at least 50% of the total kavalactones being the six major kavalactones with higher pharmacological activity. In another embodiment, the ability to produce a state of calmness, relaxation and well-being is dependent on the enhanced kava extract comprising at least 50% of the total kavalactones being the six major kavalactones with higher pharmacological activity. In another embodiment, the ability to serve as a relaxant is dependent on the enhanced kava extract comprising at least 50% of the total kavalactones being the six major kavalactones with higher pharmacological activity. In another embodiment, the ability to provide calming benefits of kava is dependent on the enhanced kava extract comprising at least 50% of the total kavalactones being the six major kavalactones with higher pharmacological activity. In another embodiment, the ability to provide relief from anxiety is dependent on the enhanced kava extract comprising at least 50% of the total kavalactones being the six major kavalactones with higher pharmacological activity. In another embodiment, the ability to provide relief from nausea is dependent on the enhanced kava extract comprising at least 50% of the total kavalactones being the six major kavalactones with higher pharmacological activity.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract serving as a relaxant.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract providing calming benefits.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract providing relief from anxiety.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract producing a state of calmness, relaxation and well-being.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract providing relief from nausea.

In one embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as poppy.

Cannabis plants produce a group of chemicals called cannabinoids, which produce mental and physical effects when consumed. Cannabis plants produce a group of chemicals that can be considered to be psychoactive components. In other words, cannabinoids can produce mental and physical effects when consumed. Both tetrahydrocannabinol (THC) and cannabidiol (CBD are cannabinoids. Owing to its ability to provide relief to an array of symptoms including pain, nausea, seizures, spasms and inflammation, the tetrahydrocannabinol (THC) containing compositions and/or cannabidiol (CBD) containing compositions of the embodiments herein could be used for both medical use and recreational use; they could be used to treat chronic pain and muscle spasm for medical use.

However, tetrahydrocannabinol (THC) containing compositions and/or cannabidiol (CBD) containing compositions also produce common side effects include dizziness, feeling tired, vomiting, anxiety, agitated, restlessness and hallucinations. These side effects are undesirable and uncomfortable for person consuming tetrahydrocannabinol (THC) containing compositions and/or cannabidiol (CBD) containing compositions.

In one embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC).

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC) with the kava component serving as a relaxant, providing calming benefits, providing relief from anxiety, producing a state of calmness, relaxation and well-being and provide relief from nausea.

In one embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD).

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD) with the kava component serving as a relaxant, providing calming benefits, providing relief from anxiety, producing a state of calmness, relaxation and well-being and provide relief from nausea.

In another embodiment, formulation for the beverages, aerated beverages, candies, hard and chewable gummy candies, confectionaries, and effervescent powder or tablet and dietary supplement containing enhanced kava extract and at least one cannabis containing component. The composition will also optionally contain one or more from the list including but not limited to different types of sugar, sugar substitute, sugar free substitute, corn syrup, lime, lemon, citrus fruits, citrus fruit juices high fructose corn syrup, rice syrup, gelatin, malt, sodium salt, potassium salt, food color, food flavor, natural flavors, preservatives, extracts and slices from nuts such as almond, hazelnut, cashew nuts and pistachios.

In one embodiment, the invention provides a process for preparing the beverages, aerated beverages, candies, hard and chewable gummy candies, confectionaries, and effervescent powder or tablet and dietary supplement comprising enhanced kava extract and a cannabis component. The beverages, aerated beverages, candies, hard and chewable gummy candies, confectionaries, and effervescent powder or tablet and dietary supplement comprising enhanced kava extract and a cannabis component are made by following some of the steps from the list of possible processing steps. The process comprises a list of processing steps as described below:

kava roots are obtained and ground;

extraction of the kava roots or other part of the plant to obtain enhanced kava extract using liquid extraction media (alcohols, mixtures of alcohols, supercritical CO2 (sCO2), or mixtures of alcohols and sCO2) and an extraction process (distillation, leaching, etc.);

post extraction recovery and/or concentration of the enhanced kava extract following extraction of the kava roots or other part of the plant;

filtering the post extraction recovery and/or concentration product that has undergone extraction to remove particulates, solids, components of soil and/or inorganic components of soil;

optionally, selectively removing the moisture or selectively drying the filtered post extraction recovered;

optionally, selectively drying the product that has undergone extraction and filtering leading to the formation of powder, paste and liquid forms of enhanced kava extract;

optionally, mixing the enhanced kava extract with at least one cannabis component and optionally with other ingredients such as milk thistle, Magnesium, Theanine vitamins, sugars, flavors and colors to make a first liquid mixture and then adding juice and/or juice concentrate to the first liquid mixture to make a second liquid mixture and mix thoroughly;

optionally, first mixing the enhanced kava extract and/or the supplement liquid mix containing enhanced kava extract and poppy and optionally lemon, adding the supplement dry mix optionally containing Theanine and Vitamin B and mixing until all the components are dispersed; adding the juice, juice concentrate to the dispersed mix and thoroughly mixing all the components using continuous mixing process;

optionally, for beverage fill bottles while continuously mixing thoroughly;

optionally, for candies, hard and chewable gummy candies and confectionaries, converts the liquid mixtures into different end products by removing the moisture or drying the mixture.

In one embodiment, the kava plants are extracted from the kava roots or other part of the plants using an extraction process and extraction medium. In another embodiment, enhanced kava extract from the kava roots or other parts of the plant is obtained by a variety of extraction processes using variety of extraction media or solvents that increase the amount of the six major kavalactones in the enhanced kava extract and/or increases the concentration of the six major kavalactones in the enhanced kava extract and/or provided a favorable mix of the six major kavalactones in the enhanced kava extract. In one embodiment of the present invention, the enhanced kava extract after extraction processes using extraction media is considered to be enhanced for having higher amounts of kavalactone content and/or higher concentration of kavalactone content and/or favorable compositional mix of six major kavalactones. The extraction process removes the impurities such as dirt, components of soil, inorganic components of soil, low molecular weight components, other minor constituents and at least some of the non-major six kavalactones that do not belong to the six major kavalactones.

In one embodiment, the extraction process consists or contains a combination of one extraction steps and one extracting media. In another embodiment, the extraction process consists or contains a combination of at least one extraction steps and at least one extracting media. In another embodiment, the extraction process consists or contains a combination of multiple extraction steps and multiple extracting media. In another embodiment, the extraction process consists or contains a combination of multiple extraction steps, multiple extracting media including but not limited to glycerin, glycerin-water, alcohol, alcohol-water mixture, and alcohol—glycerin mixture, acetate and mixtures thereof.

In one embodiment, the extraction process comprises an extracting media including but not limited to alcohols, mixtures of alcohols, mixture of alcohols and water, mixture of alcohols and glycerin acetone or combinations thereof. In another embodiment. In one embodiment, the extraction process comprises an extracting media including but not limited to alcohols such as ethyl alcohol, methyl alcohol, propyl alcohol, isopropyl alcohol, butyl alcohol. In another embodiment, the extraction process comprises an extracting media comprises glycerin or glycerin-water.

In one embodiment, the extraction media can be supercritical CO2 (carbon dioxide). The efficacy of supercritical carbon dioxide will depend on the selected pressure, temperature and period of time which are used for the supercritical carbon dioxide to mix with the kava charge in a sealed mixing vessel to dissolve the species targeted for removal.

In another embodiment, the extraction process consists of multiple extraction steps and multiple extracting media including supercritical CO2 and one or multiple extracting media selected from the list that includes but not limited to alcohol, glycerin, alcohol-water mixture, glycerin-water mixture, and alcohol—glycerin mixture. In another embodiment, the extraction process comprises a combination of multiple extraction steps, multiple extracting media including supercritical CO2, water, ethanol and ethanolic mixture. In one embodiment, the extraction process consists of first extraction step using ethanol or ethyl alcohol and a second extraction step using supercritical CO2. In another embodiment, the extraction process consists of multiple extraction steps using ethanol or ethyl alcohol and multiple extraction step using supercritical CO2. In another embodiment, the extraction process consists of one or multiple extraction steps using ethanol or ethyl alcohol and one or multiple extraction steps using supercritical CO2.

In one embodiment, enhanced kava extracts and at least one cannabis component and other ingredients such as juice, and/or juice concentrate, optionally milk thistle, Magnesium, vitamins, sugars, flavors and colors are mixed in a mixing vessel at about ambient temperature, mixing speed that mixes thoroughly and mixing time of at least 1 minute. In another embodiment, the enhanced kava extract and the supplement dry mix optionally containing Theanine and Vitamin B and supplement liquid mix containing enhanced kava extract and poppy and optionally lemon are mixed first and then the juice concentrate and the flavored juice concentrate are added, and all the ingredients are mixed at ambient temperature. Depending on the form of the end product as chewable gummy candies, beverages, associated confectionaries, and effervescent powder or tablet and dietary supplement, a final step converts the liquid mixtures into different end products or leaves it in liquid form.

The present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract and/or poppy with enhanced kava extract producing a state of calmness, relaxation, providing calming benefits, serving as a relaxant, providing relief from anxiety providing relief from nausea

wherein the enhanced kava extract was obtained from roots, rhizomes and/or lower part of the basal stem of the kava plant using an extraction process

wherein the extraction process consists of one or multiple extraction steps using ethanol or ethyl alcohol and one or multiple extraction steps using supercritical CO2 to produce an enhanced kava extract

wherein the enhanced kava extract consists of at least 50% of the total kavalactones being the six major kavalactones with higher pharmacological activity.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present disclosure provides a composition for beverages, aerated beverages, candies, hard and chewable gummy candies, confectionaries, and effervescent powder or tablet and dietary supplement comprising enhanced kava extract and at least one additional component of cannabis and/or hemp extract. Further, the process to prepare the composition is also disclosed according to present invention.

The enhanced kava extract produces a state of calmness, relaxation and well-being without diminishing cognitive performance or negative side effects. The disclosed composition of the present invention comprises an enhanced kava extract which is derived from kava plants or Piper methysticum.

Owing to their ability to provide relief to an array of symptoms including pain, nausea, seizures, spasms and inflammation, the Cannabis has been used for both medical use and recreation. In one embodiment of the embodiments of the invention, Thus based on their ability to provide relief to an array of symptoms including pain, nausea, seizures, spasms and inflammation, the Cannabis containing compositions of the embodiments herein could be used for both medical use and recreational use; these compositions could be used to treat chronic pain and muscle spasm for medical use but have common side effects include but not limited to dizziness, feeling tired, vomiting, anxiety, agitated, restlessness and hallucinations. These side effects are undesirable and uncomfortable for person consuming Cannabis containing compositions. These side effects are also physically debilitating and hinders person capacity to function normally.

In one embodiment, the disclosed composition according of the present invention comprises an enhanced kava extract that is more effective in diminishing the common negative side effects cannabis or hemp extract or poppy by serving as a relaxant, providing relief from anxiety, providing relief from nausea, providing calming benefits, producing a state of calmness, relaxation and well-being without diminishing cognitive performance. In another embodiment, the disclosed composition according of the present invention comprises an enhanced kava extract that diminishes the common negative side effects cannabis or hemp extract or poppy by serving as a relaxant, providing relief from nausea, providing relief from anxiety, providing calming benefits, producing a state of calmness, relaxation and well-being without diminishing cognitive performance. In another embodiment, the disclosed composition according of the present invention comprises an enhanced kava extract that suppresses the common negative side effects cannabis or hemp extract or poppy by serving as a relaxant, providing relief from anxiety, providing relief from nausea, providing calming benefits, producing a state of calmness, relaxation and well-being without diminishing cognitive performance. In another embodiment, the disclosed composition according of the present invention comprises an enhanced kava extract that relieves the common negative side effects cannabis or hemp extract or poppy by serving as a relaxant, providing relief from anxiety, providing relief from nausea, providing calming benefits, producing a state of calmness, relaxation and well-being without diminishing cognitive performance.

In this present invention, to diminish, relieve and/or suppress the undesired side effects of cannabis containing compositions which can be served for human consumption in solid forms, powder form, paste, liquid forms, other edible form or drinkable form, enhanced kava extract is added or incorporated into the compositions of beverages, aerated beverages, candies, hard and chewable gummy candies, confectionaries, and effervescent powder or tablet and dietary supplement comprising cannabis containing compositions that are consumed for both medical use and recreational use. In another embodiment of this present invention, to relieve the undesired side effects of cannabis component which can be served for human consumption in solid forms, powder form, paste, liquid forms, other edible form or drinkable form, enhanced kava extract is added or incorporated into the compositions of beverages, aerated beverages, candies, hard and chewable gummy candies, confectionaries, and effervescent powder or tablet and dietary supplement comprising hemp extract containing compositions of the embodiments that are consumed for both medical use and recreational use. In another embodiment of this present invention, to relieve the undesired side effects of cannabis component which can be served for human consumption in solid forms, powder form, paste, liquid form, other edible form or drinkable form, enhanced kava extract is added or incorporated into the compositions of beverages, aerated beverages, candies, hard and chewable gummy candies, confectionaries, and effervescent powder or tablet and dietary supplement comprising poppy containing compositions of the embodiments that are consumed for both medical use and recreational use. The enhanced kava extract serves as a relaxant, provide relief from anxiety, provide calming benefits, provide relief from nausea provide relief from nausea and produces a state of calmness, relaxation and well-being by diminishing the common negative side effects cannabis or hemp extract or poppy.

In one embodiment, the enhanced kava extract is obtained or derived from selected parts of the kava plants that include stems, roots, and rhizomes. In another embodiment, the enhanced kava extract is obtained or derived from selected parts of the kava plants that include fresh and dried roots, fresh and dried rhizomes, peeled, fresh and dried rhizomes, and basal stems.

In another embodiment, the enhanced kava extract is obtained or derived mainly from the roots of the kava plant, but the source of the enhanced kava extract is not restricted only from the roots and can also be harvested from other parts of the kava plants. In another embodiment, enhanced kava extract is obtained or derived from or prepared using lower part of the basal stem. rhizomes and/or roots to make fresh kava comprising compositions. In another embodiment, enhanced kava extract is obtained or derived from or prepared using lower part of the basal stem, rhizomes and/or roots to make fresh kava comprising compositions including beverages, aerated beverages, candies, and confectionaries. In another embodiment, enhanced kava extract is obtained or derived from or prepared using of intact roots, rhizomes, chips or powdered forms to make dried kava comprising compositions. In another embodiment, dried kava comprising compositions may be in the form of intact roots, rhizomes, chips or powdered forms

All part of kava plants contain water, starch, dietary fibers, sugar, proteins, minerals and kavalactones. Kava plants contain water, different types of starches, different types of dietary fibers, different types of sugar, different types of proteins, different types of minerals and kavalactones. The kava plants are harvested after they have matured in order for them to contain higher amounts of kavalactone or high kavalactone content. Kavalactones provide the pharmacological activities that allows the enhanced kava extract to produce or induce a state of calmness, relaxation and well-being without diminishing cognitive performance or causing negative side effects.

The kava plants used as a source for enhanced kava extract for the embodiments of the invention are selected from kava plants that has a history of safe use and contain kavalactone compositions that produce a state of calmness, relaxation and well-being without diminishing cognitive performance or causing other negative side-effects. These kava plants that are used as a source for enhanced kava extract for the embodiments of the invention have a history of safe use and provide the user with an experience of calmness, relaxation and well-being without diminishing cognitive performance or causing other negative side-effects are termed as noble kava.

Suitable kava plants for enhanced kava extract for the embodiments of the invention and identified according to the vernacular terms, are sourced from and include:

Federated States of Micronesia: Rahmwahnger;

Fiji: Damu. Dokobana loa, Dokobana vula, Honolulu, Loa kasa balavu. Loa kasa leka, Matakaro balavu, Matakaro leka, Qila balavu, Qila leka, Vula kasa balavu, Vula kasa leka, Yalu;

Hawaii: Hanakapi'ai, Hiwa, Honokane Ikl, Kumakua, Mahakea, Mapulehu, Moi, Nene, Opihikao, Pana'ewa, Papa 'Ele ele, Papa 'Ele ele Pu upu u, Papa kea; NOTE: all traditional Hawaii varieties are good for drinking, only two foreign varieties Isa and lwa grown are Tudei type.

Papua New Guinea (from Baluan Island): Kau kupwe;

Samoa: Ava La'au, Ava Lea, Ava Loa, Ava Tonga:

Solomon Islands: Melomelo;

Tonga: kava Fulufulu, kava Kula. kava Tea, Lekahina, Lekahina 'akau, Lekaku/a, Lekakula 'akau;

Vanuatu: Ahouia, Amon, Asiyai, Bir Kar, Bir Sul, Biyaj, Borogoru, Borogu, Ge gusug, Ge vemea, Ge wiswisket, Ke/al, Leay, Me/me/, Melomelo, Mie/a, Naga mfwok, O/itao. Palarasul. Palasa, Palimet, Pia, Poivota, Pua/iu, Puariki, Sese, Silese, Urukara. Generally, the roots, rhizomes and/or lower part of the basal stem of the harvested mature kava plants contain higher amounts of kavalactone or high kavalactone content and are the preferred part of the plant that is used as a source for making enhanced kava extract for the purpose of the embodiments of the invention. In one embodiment of the present invention, the kava plants are harvested after they have matured so they can be used as a source to obtain higher concentration of kavalactone content in the enhanced kava extract. In another embodiment of the present invention, the kava plants are harvested after they have matured so they can be used as a source to obtain higher amounts of kavalactone content. In another embodiment of the present invention, the kava plants are harvested after they have matured so they can be used as a source to obtain more favorable compositional mix of kavalactone in the enhanced kava extract. In one embodiment, enhanced kava extract disclosed in the present invention, the kava plants are usually harvested after a minimum time of 2 years so they can be used as a source to obtain higher amounts of kavalactone content or higher concentration of kavalactone content or favorable compositional mix of kavalactone in the enhanced kava extract. In another embodiment, enhanced kava extract disclosed in the present invention, the kava plants are usually harvested after a minimum time of 3 years, so they can be used as a source to obtain higher amounts of kavalactone content or higher concentration of kavalactone content or more favorable compositional mix of kavalactone in the enhanced kava extract. Preferably a minimum of 3 to 4 years or 5 years is desired before the plants are harvested so they can be used as a source to obtain higher amounts of kavalactone content or higher concentration of kavalactone content or favorable compositional mix of kavalactone in the enhanced kava extract.

In one embodiment, kavalactones are main source or the principal ingredients for serving as a relaxant, providing relief from anxiety, providing calming benefits, providing relief from nausea producing a state of calmness, relaxation and well-being without diminishing cognitive performance. There are different types of kavalactones present in the kava plant. Six of these kavalactones make up greater than 90 and in some cases 95% of the total kavalactones pharmacological activity. The six major kavalactones are kavain, dihydrokavain, methysticin, dihydromethysticin, yangonin, and desmethoxyyangonin.

In one embodiment, the ability to produce a state of calmness, relaxation and well-being is dependent on the quality of the enhanced kava extract. In another embodiment, the ability to serve as a relaxant is dependent on the quality of the enhanced kava extract. In another embodiment, the ability to provide calming benefits of kava is dependent on the quality of the enhanced kava extract. In another embodiment, the ability to provide relief from anxiety is dependent on the quality of the enhanced kava extract. In another embodiment, the ability to provide relief from nausea is dependent on the quality of the enhanced kava extract.

In one embodiment, the quality of the enhanced kava extract is related to the higher amounts of kavalactone content, higher concentration of kavalactone content, favorable compositional mix of kavalactone. In another embodiment, the quality of the enhanced kava extract is related to the higher amounts of six major kavalactone content, higher concentration of six major kavalactone content, and favorable compositional mix of six major kavalactones.

In one embodiment, the enhanced kava extract is considered to be enhanced for having higher amounts of kavalactone, higher concentration of kavalactone content, and favorable compositional mix of six major kavalactones. In another embodiment, the enhanced kava extract is considered to be enhanced for having higher amounts of six major kavalactone content, higher concentration of six major kavalactone content, and favorable compositional mix of six major kavalactones.

In another embodiment, the enhanced kava extract comprises higher amounts of kavalactone, higher concentration of kavalactone content, and favorable compositional mix of six major kavalactones. In another embodiment, the enhanced kava extract comprises higher amounts of six major kavalactone content, higher concentration of six major kavalactone content, and favorable compositional mix of six major kavalactones.

In one embodiment, the enhanced kava extract is considered to be enhanced for consisting of at least 50% of the kavalactones with higher pharmacological activity. In another embodiment, the enhanced kava extract is considered to be enhanced for consisting of at least 50% of the more active kavalactones with higher pharmacological activity. In another embodiment, the enhanced kava extract is considered to be enhanced for consisting of at least 50% of the six major kavalactones with higher pharmacological activity. In another embodiment, the enhanced kava extract is considered to be enhanced for consisting of at least 50% of the six major kavalactones.

In one embodiment, the enhanced kava extract is considered to be enhanced for consisting of at least 60% of the kavalactones with higher pharmacological activity. In another embodiment, the enhanced kava extract is considered to be enhanced for consisting of at least 60% of the more active kavalactones with higher pharmacological activity. In another embodiment, the enhanced kava extract is considered to be enhanced for consisting of at least 60% of the six major kavalactones with higher pharmacological activity. In another embodiment, the enhanced kava extract is considered to be enhanced for consisting of at least 60% of the six major kavalactones.

In one embodiment, the enhanced kava extract is considered to be enhanced for consisting of at least 70% of the kavalactones with higher pharmacological activity. In another embodiment, the enhanced kava extract is considered to be enhanced for consisting of at least 70% of the more active kavalactones with higher pharmacological activity. In another embodiment, the enhanced kava extract is considered to be enhanced for consisting of at least 70% of the six major kavalactones with higher pharmacological activity. In another embodiment, the enhanced kava extract is considered to be enhanced for consisting of at least 70% of the six major kavalactones.

In one embodiment, the enhanced kava extract is considered to be enhanced for consisting of at least 50% of the total kavalactones being kavalactones with higher pharmacological activity. In another embodiment, the enhanced kava extract is considered to be enhanced for consisting of at least 50% of the total kavalactones being the more active kavalactones with higher pharmacological activity. In another embodiment, the enhanced kava extract is considered to be enhanced for consisting of at least 50% of the total kavalactones being the six major kavalactones with higher pharmacological activity. In another embodiment, the enhanced kava extract is considered to be enhanced for consisting of at least 50% of the total kavalactones being the six major kavalactones.

In one embodiment, the enhanced kava extract is considered to be enhanced for consisting of at least 60% of the total kavalactones being kavalactones with higher pharmacological activity. In another embodiment, the enhanced kava extract is considered to be enhanced for consisting of at least 60% of the total kavalactones being the more active kavalactones with higher pharmacological activity. In another embodiment, the enhanced kava extract is considered to be enhanced for consisting of at least 60% of the total kavalactones being the six major kavalactones with higher pharmacological activity. In another embodiment, the enhanced kava extract is considered to be enhanced for consisting of at least 60% of the total kavalactones being the six major kavalactones.

In one embodiment, the enhanced kava extract is considered to be enhanced for consisting of at least 70% of the total kavalactones being kavalactones with higher pharmacological activity. In another embodiment, the enhanced kava extract is considered to be enhanced for consisting of at least 70% of the total kavalactones being the more active kavalactones with higher pharmacological activity. In another embodiment, the enhanced kava extract is considered to be enhanced for consisting of at least 70% of the total kavalactones being the six major kavalactones with higher pharmacological activity. In another embodiment, the enhanced kava extract is considered to be enhanced for consisting of at least 70% of the total kavalactones being the six major kavalactones.

In one embodiment, the enhanced kava extract comprises at least 50% of the kavalactones with higher pharmacological activity. In another embodiment, the enhanced kava extract comprises at least 50% of the more active kavalactones with higher pharmacological activity. In another embodiment, the enhanced kava extract comprises at least 50% of the six major kavalactones with higher pharmacological activity. In another embodiment, the enhanced kava extract comprises at least 50% of the six major kavalactones

In one embodiment, the enhanced kava extract comprises at least 60% of the kavalactones with higher pharmacological activity. In another embodiment, the enhanced kava extract comprises at least 60% of the more active kavalactones with higher pharmacological activity. In another embodiment, the enhanced kava extract comprises at least 60% of the six major kavalactones with higher pharmacological activity. In another embodiment, the enhanced kava extract comprises at least 60% of the six major kavalactones

In one embodiment, the enhanced kava extract comprises at least 50% of the total kavalactones being kavalactones with higher pharmacological activity. In another embodiment, the enhanced kava extract comprises at least 50% of the total kavalactones being the more active kavalactones with higher pharmacological activity. In another embodiment, the enhanced kava extract comprises at least 50% of the total kavalactones being the six major kavalactones with higher pharmacological activity. In another embodiment, the enhanced kava extract comprises at least 50% of the total kavalactones being the six major kavalactones.

In one embodiment, the enhanced kava extract comprises at least 60% of the total kavalactones being kavalactones with higher pharmacological activity. In another embodiment, the enhanced kava extract comprises at least 60% of the total kavalactones being the more active kavalactones with higher pharmacological activity. In another embodiment, the enhanced kava extract comprises at least 60% of the total kavalactones being the six major kavalactones with higher pharmacological activity. In another embodiment, the enhanced kava extract comprises at least 60% of the total kavalactones being the six major kavalactones.

In one embodiment, the enhanced kava extract comprises at least 70% of the total kavalactones being kavalactones with higher pharmacological activity. In another embodiment, the enhanced kava extract comprises at least 70% of the total kavalactones being the more active kavalactones with higher pharmacological activity. In another embodiment, the enhanced kava extract comprises at least 70% of the total kavalactones being the six major kavalactones with higher pharmacological activity. In another embodiment, the enhanced kava extract comprises at least 70% of the total kavalactones being the six major kavalactones.

In one embodiment, the effectiveness to produce a state of calmness, relaxation and well-being is dependent on the concentration of the six major kavalactones in the enhanced kava extract. In another embodiment, the ability to produce a state of calmness, relaxation and well-being is dependent on the concentration of the six major kavalactones in the enhanced kava extract. In another embodiment, the ability to serve as a relaxant is dependent on the concentration of the six major kavalactones in the enhanced kava extract. In another embodiment, the ability to provide calming benefits of kava is dependent on the concentration of the six major kavalactones in the enhanced kava extract. In another embodiment, the ability to provide relief from anxiety is dependent on the concentration of the six major kavalactones in the enhanced kava extract. In another embodiment, the ability to provide relief from nausea is dependent on the concentration of the six major kavalactones in the enhanced kava extract.

In one embodiment, the effectiveness to produce a state of calmness, relaxation and well-being is dependent on the amounts of six major kavalactones in the enhanced kava extract. In another embodiment, the ability to produce a state of calmness, relaxation and well-being is dependent on the amounts of six major kavalactones in the enhanced kava extract. In another embodiment, the ability to serve as a relaxant is dependent on the amounts of six major kavalactones in the enhanced kava extract. In another embodiment, the ability to provide calming benefits of kava is dependent on the amounts of six major kavalactones in the enhanced kava extract. In another embodiment, the ability to provide relief from anxiety is dependent on the amounts of six major kavalactones in the enhanced kava extract. In another embodiment, the ability to provide relief from nausea is dependent on the amounts of six major kavalactones in the enhanced kava extract.

In one embodiment, the effectiveness to produce a state of calmness, relaxation and well-being is dependent on the favorable compositional mix of the six major kavalactones in the enhanced kava extract. In another embodiment, the ability to produce a state of calmness, relaxation and well-being is dependent on favorable compositional mix of the six major kavalactones in the enhanced kava extract. In another embodiment, the ability to serve as a relaxant is dependent on the amounts of six major kavalactones in the enhanced kava extract. In another embodiment, the ability to provide calming benefits of kava is dependent on the favorable compositional mix of the six major kavalactones in the enhanced kava extract. In another embodiment, the ability to provide relief from anxiety is dependent on the favorable compositional mix of the six major kavalactones in the enhanced kava extract. In another embodiment, the ability to provide relief from nausea is dependent on the favorable compositional mix of the six major kavalactones in the enhanced kava extract.

In one embodiment, the effectiveness to produce a state of calmness, relaxation and well-being is dependent on the enhanced kava extract comprising at least 50% of the total kavalactones being kavalactones with higher pharmacological activity. In another embodiment, the ability to produce a state of calmness, relaxation and well-being is dependent on the enhanced kava extract comprising at least 50% of the total kavalactones being the more active kavalactones with higher pharmacological activity. In another embodiment, the ability to serve as a relaxant is dependent on the enhanced kava extract comprising at least 50% of the total kavalactones being the more active kavalactones with higher pharmacological activity. In another embodiment, the ability to provide calming benefits of kava is dependent on the enhanced kava extract comprising at least 50% of the total kavalactones being kavalactones with higher pharmacological activity. In another embodiment, the ability to provide relief from anxiety is dependent on the enhanced kava extract comprising at least 50% of the total kavalactones being kavalactones with higher pharmacological activity. In another embodiment, the ability to provide relief from nausea is dependent on the enhanced kava extract comprising at least 50% of the total kavalactones being kavalactones with higher pharmacological activity.

In one embodiment, the effectiveness to produce a state of calmness, relaxation and well-being is dependent on the enhanced kava extract comprising at least 50% of the total kavalactones being kavalactones with higher pharmacological activity. In another embodiment, the ability to produce a state of calmness, relaxation and well-being is dependent on the enhanced kava extract comprising at least 50% of the total kavalactones being the more active kavalactones with higher pharmacological activity. In another embodiment, the ability to serve as a relaxant is dependent on the enhanced kava extract comprising at least 50% of the total kavalactones being the more active kavalactones with higher pharmacological activity. In another embodiment, the ability to provide calming benefits of kava is dependent on the enhanced kava extract comprising at least 50% of the total kavalactones being the more active kavalactones with higher pharmacological activity. In another embodiment, the ability to provide relief from anxiety is dependent on the enhanced kava extract comprising at least 50% of the total kavalactones being the more active kavalactones with higher pharmacological activity. In another embodiment, the ability to provide relief from nausea is dependent on the enhanced kava extract comprising at least 50% of the total kavalactones being the more active kavalactones with higher pharmacological activity.

In one embodiment, the effectiveness to produce a state of calmness, relaxation and well-being is dependent on the enhanced kava extract comprising at least 50% of the total kavalactones being the six major kavalactones with higher pharmacological activity. In another embodiment, the ability to produce a state of calmness, relaxation and well-being is dependent on the enhanced kava extract comprising at least 50% of the total kavalactones being the six major kavalactones with higher pharmacological activity. In another embodiment, the ability to serve as a relaxant is dependent on the enhanced kava extract comprising at least 50% of the total kavalactones being the six major kavalactones with higher pharmacological activity. In another embodiment, the ability to provide calming benefits of kava is dependent on the enhanced kava extract comprising at least 50% of the total kavalactones being the six major kavalactones with higher pharmacological activity. In another embodiment, the ability to provide relief from anxiety is dependent on the enhanced kava extract comprising at least 50% of the total kavalactones being the six major kavalactones with higher pharmacological activity. In another embodiment, the ability to provide relief from nausea is dependent on the enhanced kava extract comprising at least 50% of the total kavalactones being the six major kavalactones with higher pharmacological activity.

In one embodiment, the effectiveness to produce a state of calmness, relaxation and well-being is dependent on the enhanced kava extract comprising at least 50% of the total kavalactones being the six major kavalactones. In another embodiment, the ability to produce a state of calmness, relaxation and well-being is dependent on the enhanced kava extract comprising at least 50% of the total kavalactones being the six major kavalactones. In another embodiment, the ability to serve as a relaxant is dependent on the enhanced kava extract comprising at least 50% of the total kavalactones being the six major kavalactones. In another embodiment, the ability to provide calming benefits of kava is dependent on the enhanced kava extract comprising at least 50% of the total kavalactones being the six major kavalactones. In another embodiment, the ability to provide relief from anxiety is dependent on the enhanced kava extract comprising at least 50% of the total kavalactones being the six major kavalactones. In another embodiment, the ability to provide relief from nausea is dependent on the enhanced kava extract comprising at least 50% of the total kavalactones being the six major kavalactones.

In one embodiment, the effectiveness to produce a state of calmness, relaxation and well-being is dependent on the enhanced kava extract comprising at least 60% of the total kavalactones being kavalactones with higher pharmacological activity. In another embodiment, the ability to produce a state of calmness, relaxation and well-being is dependent on the enhanced kava extract comprising at least 60% of the total kavalactones being the more active kavalactones with higher pharmacological activity. In another embodiment, the ability to serve as a relaxant is dependent on the enhanced kava extract comprising at least 60% of the total kavalactones being the more active kavalactones with higher pharmacological activity. In another embodiment, the ability to provide calming benefits of kava is dependent on the enhanced kava extract comprising at least 60% of the total kavalactones being kavalactones with higher pharmacological activity. In another embodiment, the ability to provide relief from anxiety is dependent on the enhanced kava extract comprising at least 60% of the total kavalactones being kavalactones with higher pharmacological activity. In another embodiment, the ability to provide relief from nausea is dependent on the enhanced kava extract comprising at least 60% of the total kavalactones being kavalactones with higher pharmacological activity.

In one embodiment, the effectiveness to produce a state of calmness, relaxation and well-being is dependent on the enhanced kava extract comprising at least 60% of the total kavalactones being kavalactones with higher pharmacological activity. In another embodiment, the ability to produce a state of calmness, relaxation and well-being is dependent on the enhanced kava extract comprising at least 60% of the total kavalactones being the more active kavalactones with higher pharmacological activity. In another embodiment, the ability to serve as a relaxant is dependent on the enhanced kava extract comprising at least 60% of the total kavalactones being the more active kavalactones with higher pharmacological activity. In another embodiment, the ability to provide calming benefits of kava is dependent on the enhanced kava extract comprising at least 60% of the total kavalactones being the more active kavalactones with higher pharmacological activity. In another embodiment, the ability to provide relief from anxiety is dependent on the enhanced kava extract comprising at least 60% of the total kavalactones being the more active kavalactones with higher pharmacological activity. In another embodiment, the ability to provide relief from nausea is dependent on the enhanced kava extract comprising at least 60% of the total kavalactones being the more active kavalactones with higher pharmacological activity.

In one embodiment, the effectiveness to produce a state of calmness, relaxation and well-being is dependent on the enhanced kava extract comprising at least 60% of the total kavalactones being the six major kavalactones with higher pharmacological activity. In another embodiment, the ability to produce a state of calmness, relaxation and well-being is dependent on the enhanced kava extract comprising at least 60% of the total kavalactones being the six major kavalactones with higher pharmacological activity. In another embodiment, the ability to serve as a relaxant is dependent on the enhanced kava extract comprising at least 60% of the total kavalactones being the six major kavalactones with higher pharmacological activity. In another embodiment, the ability to provide calming benefits of kava is dependent on the enhanced kava extract comprising at least 60% of the total kavalactones being the six major kavalactones with higher pharmacological activity. In another embodiment, the ability to provide relief from anxiety is dependent on the enhanced kava extract comprising at least 60% of the total kavalactones being the six major kavalactones with higher pharmacological activity. In another embodiment, the ability to provide relief from nausea is dependent on the enhanced kava extract comprising at least 60% of the total kavalactones being the six major kavalactones with higher pharmacological activity.

In one embodiment, the effectiveness to produce a state of calmness, relaxation and well-being is dependent on the enhanced kava extract comprising at least 60% of the total kavalactones being the six major kavalactones. In another embodiment, the ability to produce a state of calmness, relaxation and well-being is dependent on the enhanced kava extract comprising at least 60% of the total kavalactones being the six major kavalactones. In another embodiment, the ability to serve as a relaxant is dependent on the enhanced kava extract comprising at least 60% of the total kavalactones being the six major kavalactones. In another embodiment, the ability to provide calming benefits of kava is dependent on the enhanced kava extract comprising at least 60% of the total kavalactones being the six major kavalactones. In another embodiment, the ability to provide relief from anxiety is dependent on the enhanced kava extract comprising at least 60% of the total kavalactones being the six major kavalactones. In another embodiment, the ability to provide relief from nausea is dependent on the enhanced kava extract comprising at least 60% of the total kavalactones being the six major kavalactones.

The amount and/or concentration of the kavalactones can be measured by various analytical techniques such as HPLC.

The enhanced kava extract from the kava plant is obtained by extraction processes using extraction media or solvents that increase the amount of the six major kavalactones in the enhanced kava extract and/or increases the concentration of the six major kavalactones in the enhanced kava extract and/or provides a favorable mix of the six major kavalactones in the enhanced kava extract and/or provides a composition with at least 50% of the total kavalactones being the six major kavalactones. In another embodiment, enhanced kava extract from the a roots, rhizomes and lower part of the basal stem are obtained by extraction processes using extraction media or solvents that increase the amount of the six major kavalactones in the enhanced kava extract and/or increases the concentration of the six major kavalactones in the enhanced kava extract and/or provides a favorable mix of the six major kavalactones in the enhanced kava extract and/or provides a composition with at least 50% of the total kavalactones being the six major kavalactones.

The enhanced kava extract from the kava plant is obtained by one or more extraction processes using one of more extraction media or solvents that increase the amount of the six major kavalactones in the enhanced kava extract and/or increases the concentration of the six major kavalactones in the enhanced kava extract and/or provides a favorable mix of the six major kavalactones in the enhanced kava extract and/or provides a composition with at least 50% of the total kavalactones being the six major kavalactones. In another embodiment, enhanced kava extract from the a roots, rhizomes and lower part of the basal stem are obtained by one of more extraction processes using one of more extraction media or solvents that increase the amount of the six major kavalactones in the enhanced kava extract and/or increases the concentration of the six major kavalactones in the enhanced kava extract and/or provides a favorable mix of the six major kavalactones in the enhanced kava extract and/or provides a composition with at least 50% of the total kavalactones being the six major kavalactones.

In one embodiment, the extraction process removes dirt, components of soil, inorganic components of soil, low molecular weight components, other minor constituents and at least some of the kavalactones that do not belong to the six major kavalactones from the kava plant part being extracted. In another embodiment, the extraction process removes dirt, components of soil, inorganic components of soil, low molecular weight components, other minor constituents and substantial amounts of the kavalactones that do not belong to the six major kavalactones from the kava plant part being extracted. Solvent purification dissolves or leaches away dirt, inorganic components of soil, low molecular weight components, minor constituents and at least some of the kavalactones that do not belong to the six major kavalactones from the kava plant part being extracted. Solvent purification using liquid, high pressure gas or supercritical fluid dissolves or leaches away dirt, inorganic components of soil, low molecular weight components, minor constituents and at least some of the kavalactone that do not belong to the six major kavalactones from the kava plant part being extracted.

In one embodiment, the selection of the extraction media and the selection of extraction process employed to obtain the enhanced kava extract determines the quality of the enhanced kava extract. In another embodiment, the selection of the extraction media and the extraction process employed to obtain the enhanced kava extract determines the yield of the enhanced kava extract that is obtained or produced from the original kava plant parts that are being extracted from the kava plant part being extracted. The yield can be defined in terms of how much enhanced kava extract with higher amounts of six major kavalactone content or what concentration of six major kavalactone content, or what is the favorable compositional mix of six major kavalactone or what percentage of the total kavalactones are being the six major kavalactones.

In another embodiment of the present invention, the enhanced kava extract after extraction processes using extraction media is considered to be enhanced for having higher amounts of kavalactone content and/or higher concentration of kavalactone content and/or favorable compositional mix of kavalactones. In another embodiment, of the present invention, the enhanced kava extract after extraction processes using extraction media is considered to be enhanced for having higher amounts of six major kavalactone content and/or higher concentration of six major kavalactone content and/or favorable compositional mix of six major kavalactones.

In one embodiment of the present invention, the enhanced kava extract obtained after extraction processes using extraction media comprises higher amounts of kavalactone content and/or higher concentration of kavalactone content and/or favorable compositional mix of kavalactones. In another embodiment of the present invention, the enhanced kava extract obtained after extraction processes using extraction media comprises higher amounts of six major kavalactone content and/or higher concentration of six major kavalactone content and/or favorable compositional mix of six major kavalactones.

In another embodiment, of the present invention, the enhanced kava extract after extraction processes using extraction media is considered to be enhanced for having at least 50% of the total kavalactones being kavalactones with higher pharmacological activity. In another embodiment, of the present invention, the enhanced kava extract after extraction processes using extraction media is considered to be enhanced for having at least 50% of the total kavalactones being the more active kavalactones with higher pharmacological activity. In another embodiment, of the present invention, the enhanced kava extract after extraction processes using extraction media is considered to be enhanced for having at least 50% of the total kavalactones being the six major kavalactones with higher pharmacological activity. In another embodiment, of the present invention, the enhanced kava extract after extraction processes using extraction media is considered to be enhanced for having at least 50% of the total kavalactones being of the six major kavalactones.

In accordance to one embodiment of the present invention, the enhanced kava extract after extraction processes using extraction media is obtained in the form of powder, paste or liquid forms of enhanced kava extract and will comprise higher amounts of six major kavalactone content, higher concentration of six major kavalactone content, or favorable compositional mix of six major kavalactones. In accordance to another embodiment of the present invention, the enhanced kava extract after extraction processes using extraction media is obtained in the form of powder, paste or liquid forms of enhanced kava extract and will contain higher amounts of six major kavalactone content, higher concentration of six major kavalactone content, or favorable compositional mix of six major kavalactones.

In accordance to another embodiment of the present invention, the enhanced kava extract after extraction processes using extraction media is obtained in the form of powder, paste or liquid forms of enhanced kava extract and will comprise at least 50% of the total kavalactones being kavalactones with higher pharmacological activity. In accordance to another embodiment of the present invention, the enhanced kava extract after extraction processes using extraction media is obtained in the form of powder, paste or liquid forms of enhanced kava extract and will comprise at least 50% of the total kavalactones being the more active kavalactones with higher pharmacological activity. In accordance to another embodiment of the present invention, the enhanced kava extract after extraction processes using extraction media is obtained in the form of powder, paste or liquid forms of enhanced kava extract and will comprise at least 50% of the total kavalactones being the six major kavalactones with higher pharmacological activity. In accordance to another embodiment of the present invention, the enhanced kava extract after extraction processes using extraction media is obtained in the form of powder, paste or liquid forms of enhanced kava extract and will comprise at least 50% of the total kavalactones being the six major kavalactones.

In accordance to one embodiment, the powder, paste or liquid forms of enhanced kava extract is more effective in their ability to produce a state of calmness, relaxation and well-being, more effective in their ability to serve as a relaxant, more effective in their ability to provide calming benefits, more effective in their ability to provide relief from anxiety and more effective in their ability to provide relief from nausea. In accordance to another embodiment, the powder, paste or liquid forms of enhanced kava extract is made by the extraction processes using variety of extraction media or solvents. In accordance to another embodiment, the powder, paste or liquid forms of enhanced kava extract is obtained from the extraction processes using variety of extraction media or solvents. In accordance to another embodiment, the powder, paste or liquid forms of enhanced kava extract comprises higher amounts of kavalactone, higher concentration of kavalactone content, and favorable compositional mix of six major kavalactones. In accordance to another embodiment, the powder, paste or liquid forms of enhanced kava extract contains higher amounts of kavalactone, higher concentration of kavalactone content, and favorable compositional mix of six major kavalactones.

In one embodiment, the powder, paste and liquid forms of enhanced kava extract are more effective in their ability to produce a state of calmness, relaxation and well-being when it is one of the ingredients of beverages, aerated beverages, candies, hard and chewable gummy candies, confectionaries, and effervescent powder or tablet and dietary supplement that comprises at least one additional component of cannabis. In another embodiment, the powder, paste and liquid forms of enhanced kava extract are more effective in their ability to serve as a relaxant when it is one of the ingredients of beverages, aerated beverages, candies, hard and chewable gummy candies, confectionaries, and effervescent powder or tablet and dietary supplement that comprises at least one additional component of cannabis. In another embodiment, the powder, paste and liquid forms of enhanced kava extract are more effective in their ability to provide calming benefits when it is one of the ingredients of beverages, aerated beverages, candies, hard and chewable gummy candies, confectionaries, and effervescent powder or tablet and dietary supplement that comprises at least one additional component of cannabis. In another embodiment, the powder, paste and liquid forms of enhanced kava extract are more effective in their ability to provide relief from anxiety when it is one of the ingredients of beverages, aerated beverages, candies, hard and chewable gummy candies, confectionaries, and effervescent powder or tablet and dietary supplement that comprises at least one additional component of cannabis. In another embodiment, the powder, paste and liquid forms of enhanced kava extract are more effective in their ability to provide relief from nausea when it is one of the ingredients of beverages, aerated beverages, candies, hard and chewable gummy candies, confectionaries, and effervescent powder or tablet and dietary supplement that comprises at least one additional component of cannabis.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract. In another embodiment, the present invention provides a composition for beverages, aerated beverages, candies, hard and chewable gummy candies, confectionaries, and effervescent powder or tablet and dietary supplement comprising enhanced kava extract and at least one additional component of cannabis. In another embodiment, the present invention provides a composition for beverages, aerated beverages, candies, hard and chewable gummy candies, confectionaries, and effervescent powder or tablet and dietary supplement comprising enhanced kava extract and at least one additional component of hemp extract.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract serving as a relaxant. In another embodiment, the present invention provides a composition for beverages, aerated beverages, candies, hard and chewable gummy candies, confectionaries, and effervescent powder or tablet and dietary supplement comprising enhanced kava extract and at least one additional component of cannabis with the enhanced kava extract serving as a relaxant. In another embodiment, the present invention provides a composition for beverages, aerated beverages, candies, hard and chewable gummy candies, confectionaries, and effervescent powder or tablet and dietary supplement comprising enhanced kava extract and at least one additional component of hemp extract with the enhanced kava extract serving as a relaxant.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract providing calming benefits. In another embodiment, the present invention provides a composition for beverages, aerated beverages, candies, hard and chewable gummy candies, confectionaries, and effervescent powder or tablet and dietary supplement comprising enhanced kava extract and at least one additional component of cannabis with the enhanced kava extract providing calming benefits. In another embodiment, the present invention provides a composition for beverages, aerated beverages, candies, hard and chewable gummy candies, confectionaries, and effervescent powder or tablet and dietary supplement comprising enhanced kava extract and at least one additional component of hemp extract with the enhanced kava extract providing calming benefits.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract providing relief from anxiety. In another embodiment, the present invention provides a composition for beverages, aerated beverages, candies, hard and chewable gummy candies, confectionaries, and effervescent powder or tablet and dietary supplement comprising enhanced kava extract and at least one additional component of cannabis with the enhanced kava extract providing relief from anxiety. In another embodiment, the present invention provides a composition for beverages, aerated beverages, candies, hard and chewable gummy candies, confectionaries, and effervescent powder or tablet and dietary supplement comprising enhanced kava extract and at least one additional component of hemp extract with the enhanced kava extract providing relief from anxiety.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract producing a state of calmness, relaxation and well-being. In another embodiment, the present invention provides a composition for beverages, aerated beverages, candies, hard and chewable gummy candies, confectionaries, and effervescent powder or tablet and dietary supplement comprising enhanced kava extract and at least one additional component of cannabis with the enhanced kava extract producing a state of calmness, relaxation and well-being. In another embodiment, the present invention provides a composition for beverages, aerated beverages, candies, hard and chewable gummy candies, confectionaries, and effervescent powder or tablet and dietary supplement comprising enhanced kava extract and at least one additional component of hemp extract with the enhanced kava extract producing a state of calmness, relaxation and well-being.

In one embodiment, the present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract with the enhanced kava extract providing relief from nausea. In another embodiment, the present invention provides a composition for beverages, aerated beverages, candies, hard and chewable gummy candies, confectionaries, and effervescent powder or tablet and dietary supplement comprising enhanced kava extract and at least one additional component of cannabis with enhanced kava extract providing relief from nausea. In another embodiment, the present invention provides a composition for beverages, aerated beverages, candies, hard and chewable gummy candies, confectionaries, and effervescent powder or tablet and dietary supplement comprising enhanced kava extract and at least one additional component of hemp extract with enhanced kava extract providing relief from nausea.

In one embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as poppy. In another embodiment, the present invention provides a composition for beverages, aerated beverages, candies, hard and chewable gummy candies, confectionaries, and effervescent powder or tablet and dietary supplement comprising enhanced kava extract and a cannabis such as poppy with the enhanced kava extract serving as a relaxant. In another embodiment, the present invention provides a composition for beverages, aerated beverages, candies, hard and chewable gummy candies, confectionaries, and effervescent powder or tablet and dietary supplement comprising enhanced kava extract and a cannabis such as poppy with the enhanced kava extract providing calming benefits. In another embodiment, the present invention provides a composition for beverages, aerated beverages, candies, hard and chewable gummy candies, confectionaries, and effervescent powder or tablet and dietary supplement comprising enhanced kava extract and a cannabis such as poppy with the enhanced kava extract providing relief from anxiety. In another embodiment, the present invention provides a composition for beverages, aerated beverages, candies, hard and chewable gummy candies, confectionaries, and effervescent powder or tablet and dietary supplement comprising enhanced kava extract and a cannabis such as poppy with the enhanced kava extract producing a state of calmness, relaxation and well-being. In another embodiment, the present invention provides a composition for beverages, aerated beverages, candies, hard and chewable gummy candies, confectionaries, and effervescent powder or tablet and dietary supplement comprising enhanced kava extract and a such as poppy with enhanced kava extract providing relief from nausea.

Cannabis plants produce a group of chemicals called cannabinoids, which produce mental and physical effects when consumed. Cannabis plants produce a group of chemicals that can be considered to be psychoactive components. In other words, cannabinoids can produce mental and physical effects when consumed. Both tetrahydrocannabinol (THC) and cannabidiol (CBD are cannabinoids. In one embodiment, CBD-rich cannabis is less psychoactive than THC rich cannabis. In another embodiment, both tetrahydrocannabinol (THC) and/or cannabidiol (CBD) provide relief to an array of symptoms including pain, nausea, seizures, spasms and inflammation. In another embodiment, both tetrahydrocannabinol (THC) containing compositions and/or cannabidiol (CBD) containing compositions provide relief to an array of symptoms including pain, nausea, seizures, spasms and inflammation. Owing to its ability to provide relief to an array of symptoms including pain, nausea, seizures, spasms and inflammation, the tetrahydrocannabinol (THC) containing compositions and/or cannabidiol (CBD) containing compositions of the embodiments herein could be used for both medical use and recreational use; they could be used to treat chronic pain and muscle spasm for medical use. However, tetrahydrocannabinol (THC) containing compositions and/or cannabidiol (CBD) containing compositions also produce common side effects include dizziness, feeling tired, vomiting, anxiety, agitated, restlessness and hallucinations. These side effects are undesirable and uncomfortable for person consuming tetrahydrocannabinol (THC) containing compositions and/or cannabidiol (CBD) containing compositions. These side effects are also physically debilitating and hinders person capacity to function normally.

Both cannabidiol (CBD) and tetrahydrocannabinol (THC) are obtained from various sources including but not limited to hemp, hemp stalk, hemp stem, hemp seed, cannabis, cannabis stalk, cannabis stem, cannabis seed, cannabis flower, poppy, poppy seed.

In one embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC).

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC) with the kava component serving as a relaxant.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC) with the kava component providing calming benefits.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC) with the kava component providing relief from anxiety.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC) with the kava component producing a state of calmness, relaxation and well-being.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as tetrahydrocannabinol (THC) with the kava component to provide relief from nausea.

In one embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD).

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD) with the kava component serving as a relaxant.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD) with the kava component providing calming benefits.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD) with the kava component providing relief from anxiety.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD) with the kava component producing a state of calmness, relaxation and well-being.

In another embodiment, the present invention provides a composition for beverages, aerated beverages, candies, hard and chewable gummy candies, confectionaries, and effervescent powder or tablet and dietary supplement comprising enhanced kava extract and a cannabis comprising chemical compound such as cannabidiol (CBD) with the kava component to provide relief from nausea.

In one embodiment, beverages, aerated beverages, candies, hard and chewable gummy candies, confectionaries, and effervescent powder or tablet and dietary supplement comprising enhanced kava extract and at least one additional component of cannabis or hemp extract in the ratio of 5 mg to 950 mg of enhanced kava extract and 1 mg to 900 mg of tetrahydrocannabinol (THC). In another embodiment, beverages, aerated beverages, candies, hard and chewable gummy candies, confectionaries, and effervescent powder or tablet and dietary supplement comprises enhanced kava extract in the form of powder, paste or liquid forms and at least one additional component of cannabis or hemp extract in the ratio of 5 mg to 950 mg of enhanced kava extract and 1 mg to 900 mg of tetrahydrocannabinol (THC). In another embodiment, beverages, aerated beverages, candies, hard and chewable gummy candies, confectionaries, and effervescent powder or tablet and dietary supplement comprises enhanced kava extract with higher concentration of the six major kavalactones and at least one additional component of cannabis or hemp extract in the ratio of 5 mg to 950 mg of enhanced kava extract and 1 mg to 900 mg of tetrahydrocannabinol (THC). In another embodiment, beverages, aerated beverages, candies, hard and chewable gummy candies, confectionaries, and effervescent powder or tablet and dietary supplement comprises enhanced kava extract with higher amounts of the six major kavalactones and at least one additional component of cannabis or hemp extract in the ratio of 5 mg to 950 mg of enhanced kava extract and 1 mg to 900 mg of tetrahydrocannabinol (THC). In another embodiment, beverages, aerated beverages, candies, hard and chewable gummy candies, confectionaries, and effervescent powder or tablet and dietary supplement comprises enhanced kava extract with favorable compositional mix of the six major kavalactones and at least one additional component of cannabis or hemp extract in the ratio of 5 mg to 950 mg of enhanced kava extract and 1 mg to 900 mg of tetrahydrocannabinol (THC). The ratios of kava and/or enhanced kava extract to THC in beverages, aerated beverages, candies, hard and chewable gummy candies, confectionaries, and effervescent powder or tablet and dietary supplement provide relief from anxiety, lowers a sense of panic, serve as a relaxant, to provide calming benefits, to produce a state of calmness, relaxation and well-being and provide relief from nausea.

In one embodiment, beverages, aerated beverages, candies, hard and chewable gummy candies, confectionaries, and effervescent powder or tablet and dietary supplement comprising enhanced kava extract and at least one additional component of cannabis or hemp extract in the ratio of 5 mg to 950 mg of enhanced kava extract and 1 mg to 900 mg of cannabidiol (CBD). In another embodiment, beverages, aerated beverages, candies, hard and chewable gummy candies, confectionaries, and effervescent powder or tablet and dietary supplement comprises enhanced kava extract in the form of powder, paste or liquid forms and at least one additional component of cannabis or hemp extract in the ratio of 5 mg to 950 mg of enhanced kava extract and 1 mg to 900 mg of cannabidiol (CBD). In another embodiment, beverages, aerated beverages, candies, hard and chewable gummy candies, confectionaries, and effervescent powder or tablet and dietary supplement comprises enhanced kava extract with higher concentration of the six major kavalactones and at least one additional component of cannabis or hemp extract in the ratio of 5 mg to 950 mg of enhanced kava extract and 1 mg to 900 mg of cannabidiol (CBD). In another embodiment, beverages, aerated beverages, candies, hard and chewable gummy candies, confectionaries, and effervescent powder or tablet and dietary supplement comprises enhanced kava extract with higher amounts of the six major kavalactones and at least one additional component of cannabis or hemp extract in the ratio of 5 mg to 950 mg of enhanced kava extract and 1 mg to 900 mg of cannabidiol (CBD). In another embodiment, beverages, aerated beverages, candies, hard and chewable gummy candies, confectionaries, and effervescent powder or tablet and dietary supplement comprises enhanced kava extract with favorable compositional mix of the six major kavalactones and at least one additional component of cannabis or hemp extract in the ratio of 5 mg to 950 mg of enhanced kava extract and 1 mg to 900 mg of cannabidiol (CBD). The ratios of enhanced kava extract to CBD in beverages, aerated beverages, candies, hard and chewable gummy candies, confectionaries, and effervescent powder or tablet and dietary supplement provide relief from anxiety, lowers a sense of panic, serve as a relaxant, to provide calming benefits, to produce a state of calmness, relaxation and well-being and provide relief from nausea.

In one embodiment, the composition for the beverages, aerated beverages, candies, hard and chewable gummy candies, confectionaries, and effervescent powder or tablet and dietary supplement comprising enhanced kava extract and at least one cannabis component. In one embodiment, the composition for the beverages, aerated beverages, candies, hard and chewable gummy candies, confectionaries, and effervescent powder or tablet and dietary supplement contains enhanced kava extract and at least one cannabis component. The composition will also optionally contain one or more from the list including but not limited to concentrated fruit juice, fruit juice, Theanine, Magnesium, Vitamins, Vitamin B, lime, lemon and milk thistle.

In one embodiment, composition for the beverages, aerated beverages, candies, hard and chewable gummy candies, confectionaries, and effervescent powder or tablet and dietary supplement contains enhanced kava extract and at least one additional cannabis containing component. The formulation will also optionally contain one or more ingredients from the list including but not limited to concentrated fruit juice, fruit juice, Theanine, Magnesium, Vitamins, Vitamin B, lime, lemon and milk thistle.

In one embodiment, the composition for the beverages, aerated beverages, candies, hard and chewable gummy candies, confectionaries, and effervescent powder or tablet and dietary supplement comprising enhanced kava extract and at least one additional of cannabis containing component. The formulation will also optionally contain one or more ingredients from the list including but not limited to different types of sugar, sugar substitute, sugar free substitute, corn syrup, high fructose corn syrup, lime, lemon, citrus fruits, citrus fruit juices, rice syrup, alcoholic beverages, gelatin, malt, sodium salt, potassium salt, food color, food flavor, natural flavors, preservatives, extracts and slices from nuts such as almond, hazelnut, cashew nuts and pistachios. In another embodiment, formulation for the beverages, aerated beverages, candies, hard and chewable gummy candies, confectionaries, and effervescent powder or tablet and dietary supplement containing enhanced kava extract and at least one cannabis containing component. The composition will also optionally contain one or more ingredients from the list including but not limited to different types of sugar, sugar substitute, sugar free substitute, corn syrup, lime, lemon, citrus fruits, citrus fruit juices high fructose corn syrup, alcoholic beverages, rice syrup, gelatin, malt, sodium salt, potassium salt, food color, food flavor, natural flavors, preservatives, extracts and slices from nuts such as almond, hazelnut, cashew nuts and pistachios.

In one embodiment, beverages, aerated beverages, candies, hard and chewable gummy candies, confectionaries, and effervescent powder or tablet and dietary supplement will contain a mixture juice concentrate, flavored juice concentrate, supplement dry mix optionally containing Theanine and Vitamin B, supplement liquid mix containing enhanced kava extract and poppy and optionally lemon. In another embodiment, the mixture contains from about 2.5 to about 3.0 gallons of juice concentrate, from about 0.8 to about 1.2 gallons of flavored juice concentrate, from about 120 grams to about 140 grams of supplement dry mix and from about 4000 to about 5000 grams of supplement liquid mix or in the same ratios of the components. In another embodiment, the mixture contains from about 2.0 to about 4.0 gallons of juice concentrate, from about 0.5 to about 1.5 gallons of flavored juice concentrate, from about 100 grams to about 160 grams of supplement dry mix and from about 3000 to about 6000 grams supplement liquid mix or in the same ratios of the components.

In one embodiment, beverages, aerated beverages, candies, hard and chewable gummy candies, confectionaries, and effervescent powder or tablet and dietary supplement will contain a mixture juice concentrate, flavored juice concentrate, supplement dry mix optionally containing Theanine and Vitamin B, enhanced kava extract and supplement liquid mix containing enhanced kava extract and poppy and optionally lemon. In another embodiment, the mixture contains from about 10 to about 60 gallons of juice concentrate, from about 5 to about 40 gallons of flavored juice concentrate, from about 2000 grams to about 4000 grams of supplement dry mix, from about 3 to about 20 gallons of supplement liquid mix and 1000 to 3000 enhanced kava extracts or in the same ratios of the components. In another embodiment, the mixture contains from about 15 to about 55 gallons of juice concentrate, from about 10 to about 35 gallons of flavored juice concentrate, from about 1000 grams to about 3000 grams of supplement dry mix, from about 5 to about 15 gallons of supplement liquid mix and 1500 to 2500 enhanced kava extract or in the same ratios of the components. All the compositions to form beverages, aerated beverages, candies, hard and chewable gummy candies, confectionaries, and effervescent powder or tablet and dietary supplement comprises powder, paste and liquid forms of enhanced kava extract formed after extraction of the kava roots or other part of the plant.

In one embodiment, the invention provides a process for preparing the beverages, aerated beverages, candies, hard and chewable gummy candies, confectionaries, and effervescent powder or tablet and dietary supplement comprising enhanced kava extract and a cannabis component. The beverages, aerated beverages, candies, hard and chewable gummy candies, confectionaries, and effervescent powder or tablet and dietary supplement comprising enhanced kava extract and a cannabis component are made by following some of the steps from the list of possible processing steps. The process comprises a list of processing steps as described below:
kava roots are obtained and ground;
extraction of the kava roots or other part of the plant to obtain enhanced kava extract using liquid extraction media (alcohols, mixtures of alcohols, supercritical CO2 (sCO2), or mixtures of alcohols and sCO2) and an extraction process (distillation, leaching, etc.);
post extraction recovery and/or concentration of the enhanced kava extract following extraction of the kava roots or other part of the plant;
filtering the post extraction recovery and/or concentration product that has undergone extraction to remove particulates, solids, components of soil and/or inorganic components of soil;
optionally, selectively removing the moisture or selectively drying the filtered post extraction recovered; optionally, selectively drying the product that has undergone extraction and filtering leading to the formation of powder, paste and liquid forms of enhanced kava extract;
optionally, mixing the enhanced kava extract with at least one cannabis component and optionally with other ingredients such as milk thistle, Magnesium, Theanine vitamins, sugars, flavors and colors to make a first liquid mixture and then adding juice and/or juice concentrate to the first liquid mixture to make a second liquid mixture and mix thoroughly;
optionally, first mixing the enhanced kava extract and/or the supplement liquid mix containing enhanced kava extract and poppy and optionally lemon, adding the supplement dry mix optionally containing Theanine and Vitamin B and mixing until all the components are dispersed; adding the juice, juice concentrate to the dispersed mix and thoroughly mixing all the components using continuous mixing process;
optionally, for beverage fill bottles while continuously mixing thoroughly;
optionally, for candies, hard and chewable gummy candies and confectionaries, converts the liquid mixtures into different end products by removing the moisture or drying the mixture.

In one embodiment, the kava plants are extracted from the kava roots or other part of the plants using an extraction process and extraction medium. In another embodiment, enhanced kava extract from the kava roots or other parts of the plant is obtained by a variety of extraction processes using variety of extraction media or solvents that increase the amount of the six major kavalactones in the enhanced kava extract and/or increases the concentration of the six major kavalactones in the enhanced kava extract and/or provides a favorable mix of the six major kavalactones in the enhanced kava extract and/or provides a composition with at least 50% of the total kavalactones being the six major kavalactones with higher pharmacological activity. In one embodiment of the present invention, the enhanced kava extract after extraction processes using extraction media is considered to be enhanced for having higher amounts of kavalactone content and/or higher concentration of kavalactone content and/or favorable compositional mix of six major kavalactones and/or provides a composition with at least 50% of the total kavalactones being the six major kavalactones with higher pharmacological activity. The extraction process removes the impurities such as dirt, components of soil, inorganic components of soil, low molecular weight components, other minor constituents and at least some of the non-major six kavalactones that do not belong to the six major kavalactones. Exemplary extraction processes include but not limited to solvent purification, solvent extraction, distillation, cold distillation, high pressure gas, supercritical fluid extraction and combinations thereof. The product of the extraction process can be in the form of powder, paste and liquid forms of enhanced kava extract.

In one embodiment, the extraction process comprises a combination of one extraction steps and one extracting media. In another embodiment, the extraction process comprises a combination of at least one extraction steps and at least one extracting media. In another embodiment, the extraction process comprises a combination of multiple extraction steps and multiple extracting media. In another embodiment, the extraction process comprises a combination of multiple extraction steps, multiple extracting media including but not limited to glycerin, glycerin-water, alcohol, alcohol-water mixture, and alcohol—glycerin mixture, acetate and mixtures thereof.

In one embodiment, the extraction process consists or contains a combination of one extraction steps and one extracting media. In another embodiment, the extraction process consists or contains a combination of at least one extraction steps and at least one extracting media. In another embodiment, the extraction process consists or contains a combination of multiple extraction steps and multiple extracting media. In another embodiment, the extraction process consists or contains a combination of multiple extraction steps, multiple extracting media including but not limited to glycerin, glycerin-water, alcohol, alcohol-water mixture, and alcohol—glycerin mixture, acetate and mixtures thereof.

The extraction media can be a liquid such as but not limited to alcohols, mixtures of alcohols, mixture of alcohols and water, glycerin, mixture of glycerin and water acetone, acetate or combinations thereof. The efficacy of the extraction process will depend on the boiling point of the extraction media, the concentrations of the initial kava content in the extraction media in the extraction media, the concentrations of the initial kava plant content in the extraction media, ratio of kava pant content to extraction media, temperature, the agitation time which is the mixing time of the kava with the liquid extraction media, the mixing intensity or the mixing speed of the agitation process and the various filtration techniques.

In one embodiment, the extraction process comprises an extracting media including but not limited to alcohols, mixtures of alcohols, mixture of alcohols and water, mixture of alcohols and glycerin acetone or combinations thereof. In another embodiment. In one embodiment, the extraction process comprises an extracting media including but not limited to alcohols such as ethyl alcohol, ethanol, methyl alcohol, propyl alcohol, isopropyl alcohol, butyl alcohol or combinations thereof. In one embodiment, the extraction process comprises ethyl alcohol or ethanol. In another embodiment, the extraction process comprises an extracting media comprises glycerin or glycerin-water. In another embodiment, the extraction process comprises an extracting media comprises acetate. In another embodiment, the extraction process comprises an extracting media comprises acetone.

In one embodiment, the extracting media comprises of Glycerin and Glycerin/water mixture. In another embodiment, the extracting media comprises of alcohol/water mixture in the ratio of 20/80, 30/70, 40/60, 50/50, 60/40, 75/25, 80/20. In another embodiment, the extracting media comprises alcohol-glycerin mixture in the ratio of 20/80, 30/70, 40/60, 50/50. In another embodiment, the extracting media comprises different alcohols.

In one embodiment, the extraction can be carried out in the temperature range of 60 to 180° F. In one embodiment, the extraction can be carried out in the temperature range of 70 to 160° F. In another embodiment, the extraction can be carried out in the temperature range of 80 to 150° F. In another embodiment, the extraction can be carried out in the temperature range of 120 to 150° F. In another embodiment, the extraction can be carried out in the temperature range of 125 to 145° F.

In one embodiment, the extraction time is between 60 and 150 minutes. In another embodiment, extraction time is between 70 and 120 minutes. In another embodiment, extraction time is between 80 and 110 minutes.

In one embodiment, the extraction media can be supercritical CO2 (carbon dioxide). The efficacy of supercritical carbon dioxide will depend on the selected pressure, temperature and period of time which are used for the supercritical carbon dioxide to mix with the kava charge in a sealed mixing vessel to dissolve the species targeted for removal. The high-pressure stream of gas plus material that has been dissolved at the particular pressure level for the fraction is passed through the pressure reduction valve. When the pressure is reduced to 1 atmosphere across the valve, the dissolved material precipitates as a fraction and collects in a filter flask. Multiple fractions can be obtained depending on the amount of time that the high-pressure stream of gas plus material is allowed to come out of the sealed mixing vessel; lowest molecular weight species or impurities come out first and with passage of time, as higher molecular weight species are removed leaving behind an enhanced kava extract that is of high quality, purity, having higher concentration of the six major kavalactones and more effective.

In another embodiment, the extraction process comprises an extracting media comprises supercritical CO2 (carbon dioxide). In one embodiment, the extracting media comprises supercritical CO2 with process conditions that will remove at least one low molecular weight fraction. In another embodiment, the extracting media comprises supercritical CO2 with process conditions that will remove at least two low molecular weight fractions.

In one embodiment, the extraction process consists of multiple extraction steps and multiple extracting media including supercritical CO2 and at least one alcohol-water mixtures. In another embodiment, the extraction process consists of multiple extraction steps and multiple extracting media including supercritical CO2 and glycerin or glycerin-water mixture.

In one embodiment, the extraction process consists of first extraction step using ethanol or ethyl alcohol and a second extraction step using supercritical CO2. In another embodiment, the extraction process consists of multiple extraction steps using ethanol or ethyl alcohol and multiple extraction step using supercritical CO2. In another embodiment, the extraction process consists of one or multiple extraction steps using ethanol or ethyl alcohol and one or multiple extraction steps using supercritical CO2.

In another embodiment, the extraction process consists of multiple extraction steps and multiple extracting media including supercritical CO2 and one or multiple extracting media selected from the list that includes but not limited to alcohol, glycerin, alcohol-water mixture, glycerin-water mixture, and alcohol—glycerin mixture, acetate or mixtures thereof. In another embodiment, the extraction process comprises a combination of multiple extraction steps, multiple extracting media including supercritical CO2, water, ethanol and ethanolic mixture.

In one embodiment, the yield of the enhanced kava extract is how much enhanced kava extract is obtained or produced from the original kava plant parts that are being extracted from the kava plant part being extracted. In another embodiment, the yield of the enhanced kava extract is how much enhanced kava extract with higher amounts of six major kavalactone content, higher concentration of six major kavalactone content, or favorable compositional mix of six major kavalactone is obtained or produced from the original kava plant parts that are being extracted from the kava plant part being extracted.

In one embodiment, the extraction process produces an extraction yield of 5 to 60% by weight based on original amount of kava that was used in extraction. In another embodiment, the extraction process produces an extraction yield of 10 to 50% by weight based on original amount of kava plant part that was used for extraction. In another embodiment, the extraction process produces an extraction yield of 20 to 50% by weight based on original amount of kava plant part that was used for extraction. In another embodiment, the extraction process produces an extraction yield of 15 to 55% by weight based on original amount of kava plant part that was used for extraction. In another embodiment, the extraction process produces an extraction yield by weight based on original amount of kava plant part that was used for extraction which was greater than 5%. In another embodiment, the extraction process produces an extraction yield by weight based on original amount of kava plant part that was used for extraction which was greater than 20%. In another embodiment, the extraction process produces an extraction yield by weight based on original amount of kava plant part that was used for extraction which was greater than 30%. In another embodiment, the extraction process produces an extraction yield by weight based on original amount of kava plant part that was used for extraction which was greater than 40%.

The post extraction recovery and/or concentration process involves filtering of the product that has undergone extraction. Filtering the product that has undergone extraction removes particulates, solids, components of soil and/or inorganic components of soil. The process involves a number or includes but not limited to filtering, filtering assisted with vacuum, filtering at elevated temperature, drying to remove moisture and drying in oven to at least partially removing the moisture or reducing the amount of moisture. In accordance with the level of moisture, the enhanced kava extract after drying can be in the form of powder, paste or liquid forms,

In one embodiment, enhanced kava extracts and at least one cannabis component and other ingredients such as juice, and/or juice concentrate, optionally milk thistle, Magnesium, vitamins, sugars, flavors and colors are mixed in a mixing vessel at about ambient temperature, mixing speed that mixes thoroughly and mixing time of at least 1 minute. In another embodiment, the enhanced kava extract and the supplement dry mix optionally containing Theanine and Vitamin B and supplement liquid mix containing enhanced kava extract and poppy and optionally lemon are mixed first and then the juice concentrate and the flavored juice concentrate are added, and all the ingredients are mixed at ambient temperature. Depending on the form of the end product as chewable gummy candies, beverages, associated confectionaries, and effervescent powder or tablet and dietary supplement, a final step converts the liquid mixtures into different end products or leaves it in liquid form.

The present invention provides a composition for beverages, aerated beverages, candies, hard and chewable gummy candies, confectionaries, and effervescent powder or tablet and dietary supplement comprising enhanced kava extract and at least one additional component of cannabis and/or hemp extract and/or poppy with enhanced kava extract producing a state of calmness, relaxation, providing calming benefits, serving as a relaxant, providing relief from anxiety providing relief from nausea wherein the enhanced kava extract was obtained from roots, rhizomes and/or lower part of the basal stem of the kava plant using an extraction process wherein the extraction process consists of one or multiple extraction steps using ethanol or ethyl alcohol and one or multiple extraction steps using supercritical CO2 to produce an enhanced kava extract wherein the enhanced kava extract consists of at least 50% of the total kavalactones being the six major kavalactones with higher pharmacological activity.

### Example 1

A relaxation drink produced with 4 gallons of concentrated juice mix, 4 Kg of supplement dry mix that contains Theanine, enhanced kava extract, Magnesium, Vitamin B, and milk thistle, and 32 oz. of poppy extract.

### Example 2

A relaxation drink produced with 12 gallons of concentrated juice mix, 500 gm of supplement dry mix that contains Theanine, Vitamin B, and milk thistle, and 359 oz. of poppy and enhanced kava extract.

### Example 3

A relaxation drink produced with 7 gallons of concentrated juice mix, 280 gm of supplement dry mix that contains Theanine, and milk thistle, and 230 oz. of liquid mix (poppy and enhanced kava extract).

### Example 4

A relaxation drink produced with 25 gallons of concentrated juice mix, 17 gallons of additional flavor juice concentrate, 1000 gm of supplement dry mix that contains Theanine, and Vitamin B, and 5.5 gallons of supplemental liquid mix (poppy, enhanced kava extract and lemon).

### Example 5

A relaxation drink produced with 50 gallons of concentrated juice mix, 33 gallons of additional flavor juice concentrate, 2000 gm of supplement dry mix that contains Theanine, and Vitamin B, and 10 gallons of supplemental liquid mix (poppy, enhanced kava extract and lemon), 80 oz. of enhanced kava extract.

### Example 6

A relaxation drink produced with 20 gallons of concentrated juice mix, 15 gallons of additional flavor juice concentrate, 500 gm of supplement dry mix that contains Theanine, and Vitamin B, and 7.5 gallons of supplemental liquid mix (poppy, enhanced kava extract and lemon), 59 oz. of enhanced kava extract.

1. A human edible or drinkable product comprising an enhanced kava extract, wherein the enhanced kava extract comprises a kava product consisting of a kava extract that is extracted using a material comprising CO2 and/or ethanol extraction method from a selected part of a kava plant and containing 50% or more of a kavalactone as measured by HPLC, wherein the kava plant is not a wild type kava such that the kava product is safe for human consumption, wherein the human edible or drinkable product further comprises an additive comprising a tetrahydrocannabinol extract, a cannabidiol extract, a hemp extract or combinations thereof, wherein a ration of the enhanced kava extract to the tetrahydrocannabinol extract, the cannabidiol extract or the combinations thereof provides relief from anxiety, lowers a sense of panic and serves as a relaxant to provide calming benefits, to produce a state of calmness, relaxation and well-being, and to provide relief from nausea to a person consuming the human edible or drinkable product.

2. The human edible or drinkable product of claim 1, wherein the additive consists essentially of the tetrahydrocannabinol extract.

3. The human edible or drinkable product of claim 1, wherein the additive consists essentially of the cannabidiol extract.

4. The human edible or drinkable product of claim 1, wherein the additive consists essentially of the hemp extract.

5. The human edible or drinkable product of claim 1, wherein the human edible or drinkable product provides a benefit of reduction in an incidence or severity of anxious feelings or panic sometimes associated with the tetrahydrocannabinol.

6. The human edible or drinkable product of claim 1, wherein the human edible or drinkable product is selected from a group consisting of a hard candy, a chewable candy, a beverage, a dietary supplement, and a combination thereof.

7. The human edible or drinkable product of claim 6, wherein the hard candy, the chewable candy, the beverage or the dietary supplement comprises about 5 mg-950 mg of the enhanced kava extract and about 1 mg-900 mg of a tetrahydrocannabinol extract in a total candy.

8. The human edible or drinkable product of claim 7, wherein the hard candy, the chewable candy, the beverage or the dietary supplement comprises about 5 mg-950 mg of the enhanced kava extract in the total candy and about 1 mg-900 mg of a cannabidiol extract in the total candy.

9. The human edible or drinkable product of claim 7, wherein the hard candy, the chewable candy, the beverage or the dietary supplement comprises about 5 mg-950 mg of the enhanced kava extract in the total candy and about 1 mg-900 mg of a hemp extract in the total candy.

10. The human edible or drinkable product of claim 1, wherein the selected part of the kava plant comprises a lower part of a basal stem, a rhizomes and/or a roots.

11. The human edible or drinkable product of claim 1, wherein the material comprises supercritical CO2.

12. The human edible or drinkable product of claim 1, wherein the material comprises supercritical CO2 and ethanol.

13. The human edible or drinkable product of claim 1, wherein the material consists essentially of supercritical CO2.

14. The human edible or drinkable product of claim 1, wherein the material consists essentially of ethanol.

15. The human edible or drinkable product of claim 1, wherein the enhanced kava extract contains 60% or more of the kavalactone as measured by HPLC.

16. A human edible or drinkable product comprising an enhanced kava extract, wherein the enhanced kava extract comprises a kava product consisting of a kava extract that is extracted using a non-organic solvent from a selected part of a kava plant and containing 50% or more of a kavalactone as measured by HPLC, wherein the kava plant is not a wild type kava such that the kava product is safe for human consumption wherein the human edible or drinkable product further comprises an additive comprising a tetrahydrocannabinol extract, a cannabidiol extract, a hemp extract or combinations thereof, wherein a ratio of the enhanced kava extract to the tetrahydrocannabinol extract, the cannabidiol extract, or the combinations thereof provides relief from anxiety, lowers a sense of panic and serves as a relaxant to provide calming benefits, to produce a state of calmness, relaxation and well-being, and to provide relief from nausea to a person consuming the human edible or drinkable product.

17. The human edible or drinkable product of claim 16, wherein the additive consists essentially of the tetrahydrocannabinol extract.

18. The human edible or drinkable product of claim 16, wherein the additive consists essentially of the cannabidiol extract.

19. The human edible or drinkable product of claim 16, wherein the additive consists essentially of the hemp extract.

20. The human edible or drinkable product of claim 16, wherein the human edible or drinkable product provides a benefit of reduction in an incidence or severity of anxious feelings or panic sometimes associated with the tetrahydrocannabinol.

21. The human edible or drinkable product of claim 16, wherein the human edible or drinkable product is selected from a group consisting of a hard candy, a chewable candy, a beverage, a dietary supplement, and a combination thereof.

22. The human edible or drinkable product of claim 21, wherein the hard candy, the chewable candy, the beverage or the dietary supplement comprises about 5 mg-950 mg of the enhanced kava extract and about 1 mg-900 mg of a tetrahydrocannabinol extract in a total candy.

23. The human edible or drinkable product of claim 22, wherein the hard candy, the chewable candy, the beverage or the dietary supplement comprises about 5 mg-950 mg of the enhanced kava extract in the total candy and about 1 mg-900 mg of a cannabidiol extract in the total candy.

24. The human edible or drinkable product of claim 22, wherein the hard candy, the chewable candy, the beverage or the dietary supplement comprises about 5 mg-950 mg of the enhanced kava extract in the total candy and about 1 mg-900 mg of a hemp extract in the total candy.

25. The human edible or drinkable product of claim 16, wherein the selected part of the kava plant comprises a lower part of a basal stem, a rhizome and/or a roots.

26. The human edible or drinkable product of claim 1, wherein the enhanced kava extract contains 60% or more of the kavalactone as measured by HPLC.

27. The human edible or drinkable product of claim 16, wherein the non-organic solvent comprises supercritical CO2.

28. The human edible or drinkable product of claim 16, wherein the non-organic solvent consists essentially of supercritical CO2.
